(19) United States
(12) Patent Application Publication
(10) Pub. No.: US 2013/0053709 A1
(43) Pub. Date: Feb. 28, 2013

(54) ARM ROLLING STRIP FOR SPHYGMOMANOMETERS

(71) Applicant: Daniel C.M. TSENG, New Taipei City (TW)

(72) Inventor: Daniel C.M. TSENG, New Taipei City (TW)

(21) Appl. No.: 13/222,923

(22) Filed: Aug. 31, 2011

(51) Int. Cl. A61B 5/02

(52) U.S. Cl. 600/499

(57) ABSTRACT

An arm rolling strip for sphygmomanometers includes an elastic first rolling strip and an elastic second rolling strip wrapping partially on the first rolling strip. The first rolling strip has an aperture holding an air nozzle communicating therewith. The second rolling strip has a guide track formed on the arched surface thereof relative to the air nozzle to restrict sliding of the second rolling strip on the arched surface of the first rolling strip. The arm rolling strip thus formed can be extended to fit an adult's thicker arm or shortened to fit a child's thinner arm to provide much comfort and fitness to users when in use.

### FIELD OF THE INVENTION

The present invention relates to a medical measurement accessory and particularly to an arm rolling strip used to measure people's blood pressure.

### BACKGROUND OF THE INVENTION

China patent No. CN1200638C entitled “Arm cover for blood pressure monitor” discloses a technique which provides an arm cover for a blood pressure monitor equipped with an elastic coiled member. It can be easily fastened to a user's arm without hurting people and offer desired adaptability. The elastic coiled member has one end extendable outwards and another end coiling inwards. More specifically, one end and another end are positioned opposing each other. The curvature radius increases gradually from the opposing location to the one end. Another China patent No. CN1233296C entitled “Arm lacing for sphygmomanometer” discloses an arm lacing which is adaptable to various arm profiles for measuring blood pressure, especially desirable for arms slanted at a greater degree. The arm lacing contains an elastic plate inside that surrounds a user's arm and provides varying axial rigidity. The portion with higher rigidity can securely wrap user's arm while the lower rigidity portion matches user's arm snugly, hence the arm lacing is adaptable to arms with varying shapes and sizes.

The aforesaid conventional techniques include an elastic member (or elastic plate) coiling inwards aimed to be versatile and adaptable to arms of varying profiles so that it can fit the thin and thick portions of the arm at the same time. To meet this purpose the curvature radius or rigidity at two ends of the elastic member are different. They do not take into account of the great size differences of adult and children's arms. In practice, if the elastic member is made in a smaller curvature radius or higher rigidity, it wraps the thicker arm of an adult too tight and causes great discomfort when in use. On the other hand, if the elastic member is made in a larger curvature radius or lower rigidity, it wraps the thinner arm of a child too loose and cannot fully wrap the arm and causes inaccurate measurement, then an extra pressure has to be applied on the elastic member. The elastic member loses the function it should provide. All this shows that how to make the elastic member fits adult's thicker arms and children's thinner arms at the same time is a big issue in design and industry yet to be resolved.

### SUMMARY OF THE INVENTION

The primary object of the present invention is to solve the disadvantages of the conventional techniques by providing a product adaptable to adult's thicker arms and children's thinner arms in a snug manner.

To achieve the foregoing object the invention provides an arm rolling strip for sphygmomanometers that includes an elastic first rolling strip and an elastic second rolling strip wrapping partially on the first rolling strip. The first rolling strip has an aperture holding an air nozzle communicating therewith. The second rolling strip has a guide track formed on the arched surface thereof relative to the air nozzle to restrict sliding of the second rolling strip on the arched surface of the first rolling strip.

By means of the features set forth above the second rolling strip can be moved relative to the first rolling strip and is retained in the guide track through the air nozzle. The arm rolling strip thus formed is extendable to suit an adult's thicker arm and retractable to suit a child's thinner arm. Compared with the conventional techniques the invention provides more comfort for users during measurement of blood pressure and also fits better.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a perspective view of a first embodiment of the invention.

FIG. 2 is an exploded view according to FIG. 1.

FIGS. 3A and 3B are schematic sectional views according to FIG. 1 in use conditions.

FIGS. 4A and 4B are schematic views of another embodiment of the guide track according to FIG. 1 in use conditions.

FIG. 5 is a schematic view of adding an isolation cover according to FIG. 1.

FIGS. 6A and 6B are schematic sectional views according to FIG. 5 in use conditions.

FIG. 7 is a perspective view of a second embodiment of the invention.

FIGS. 8A and 8B are schematic views of another embodiment of auxiliary track according to FIG. 7 in use conditions

### DETAILED DESCRIPTION OF THE PREFERRED EMBODIMENTS

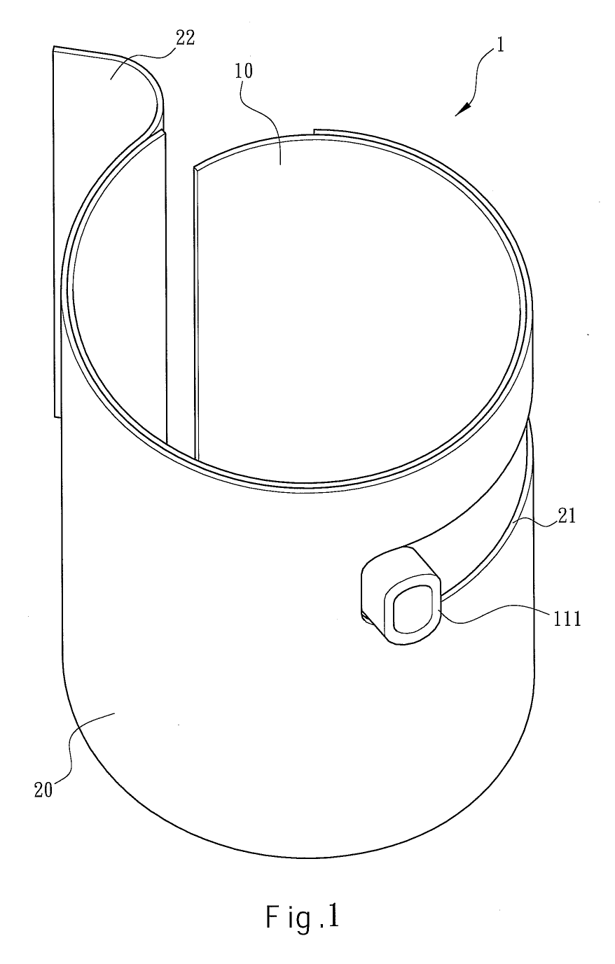

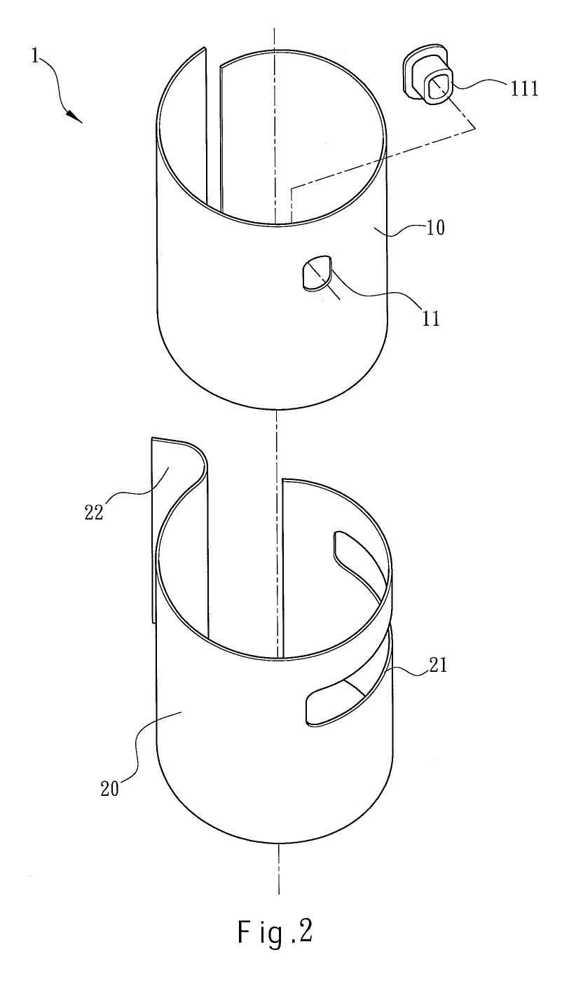

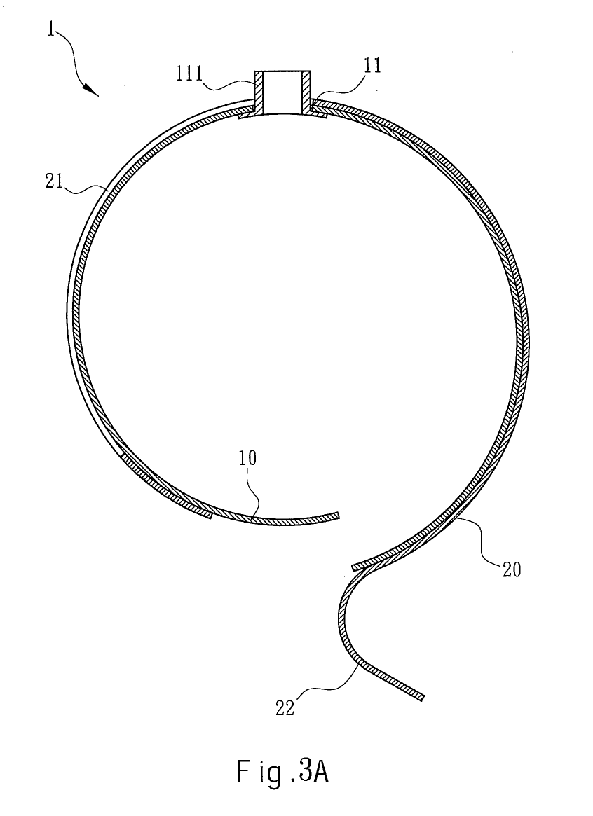

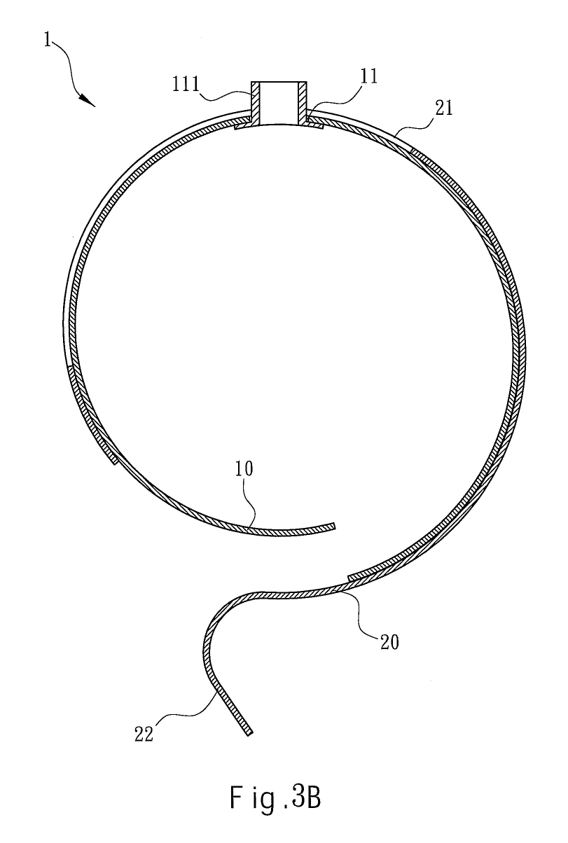

Please referring to FIGS. 1, 2 and 3A, the present invention aims to provide an arm rolling strip 1 for sphygmomanometers. It includes an elastic first rolling strip 10 and an elastic second rolling strip 20 wrapping partially on the first rolling strip 10. The first rolling strip 10 has an aperture 11 holding an air nozzle 111 communicating therewith. The second rolling strip 20 has a guide track 21 formed on the arched surface thereof relative to the air nozzle 111 to restrict sliding of the second rolling strip 20 on the arched surface of the first rolling strip 10. The second rolling strip 20 has a distal end 22 extended therefrom in the direction opposite to the rolling direction thereof, or in the tangent direction of the rolling direction of the second rolling strip 20, or may also be in any direction between the tangent direction of the rolling direction and the direction opposite to the rolling direction of the second rolling strip 20. By exerting a force on the distal end 22, the second rolling strip 20 can slide on the arched surface of the first rolling strip 10 in virtue of the restriction of the guide track 21. Moreover, the first rolling strip 10 is preferably rolled at a gradually increasing curvature radius. When the second rolling strip 20 is located at a position relative to the first rolling strip 10 as shown in FIG. 3A, the arm rolling strip 1 has a smaller curvature radius, and its total rigidity is higher because overlapping portion between the first and second rolling strips 10 and 20 is greater, hence it is suitable to fit a child's thinner arm and provides full wrapping. When the positional relationship between the second rolling strip 20 and first rolling strip 10 is changed as shown in FIG. 3B, although the lacing plate 1 still maintains the same curvature radius, total rigidity of the arm rolling strip 1 decreases because the overlapping portion between the first and second rolling strips 10 and 20 is smaller, but total length of the arm rolling strip 1 increases. Hence when the arm rolling strip 1 wraps an adult's thicker arm, it is expanded even with a smaller curvature radius and becomes longer at a lower rigidity to better fit the adult's thicker arm and provides full wrapping without wrapping too tight or causing discomfort. Thus it is adaptable to children's thinner arm and adult's thicker arm and also provides better fit for blood pressure measurement.

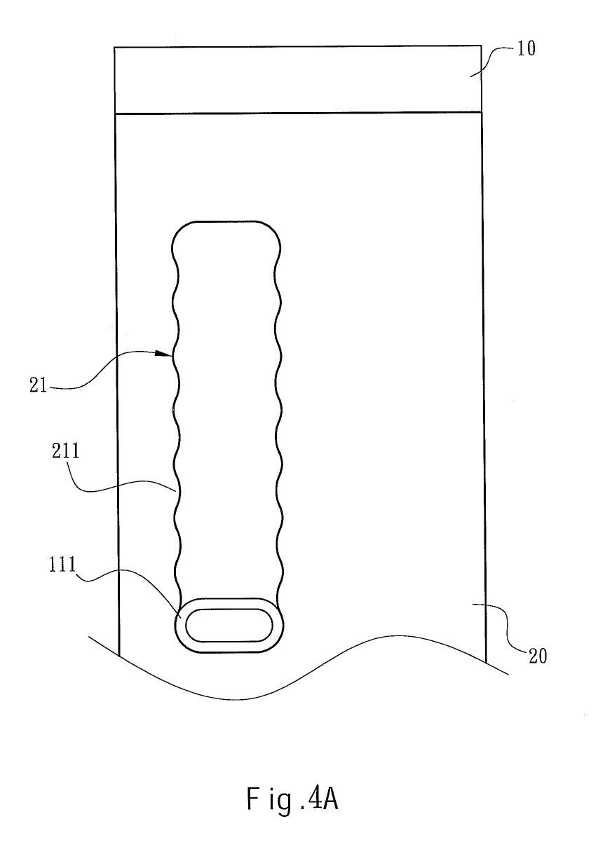

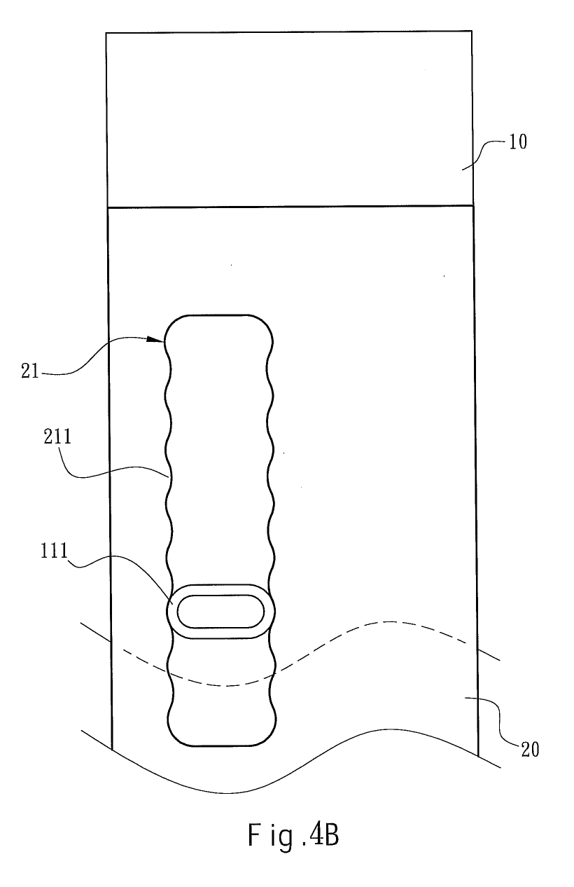

Please also refer to FIGS. 4A and 4B for another embodiment of the guide track 21. The guide track 21 in this embodiment has a plurality of positioning teeth 211 on at least one longer side, hence the air nozzle 111 can be positioned by stages on the guide track 21.

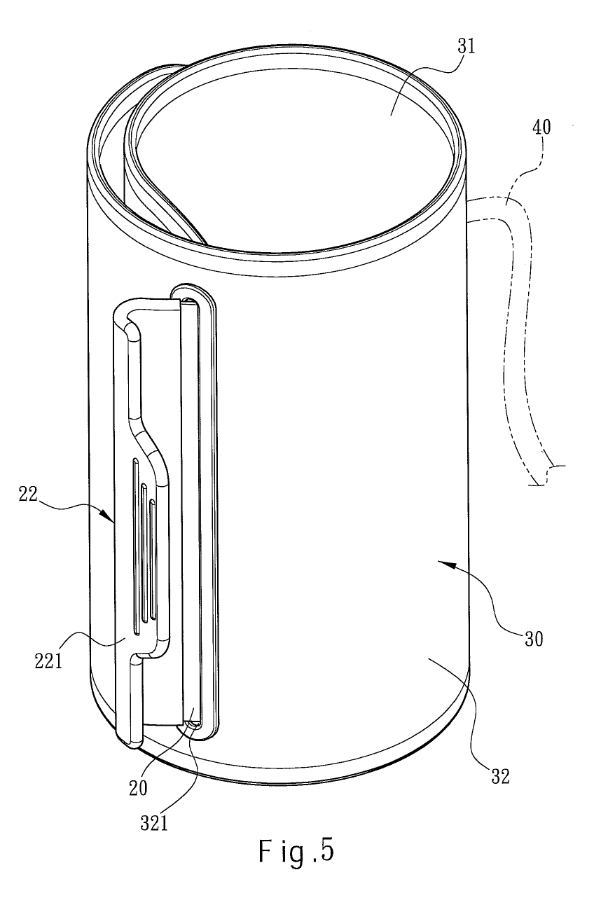

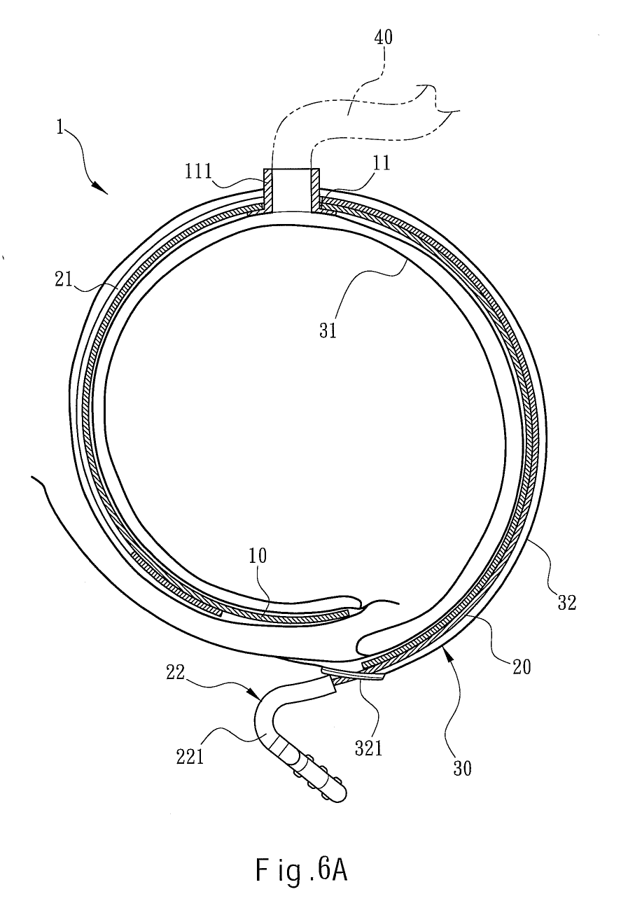

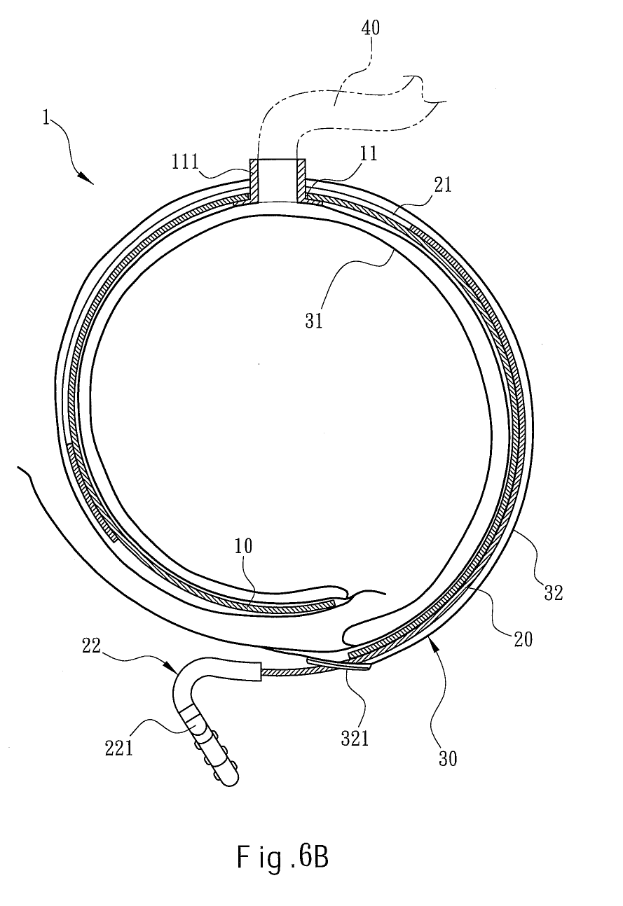

Referring to FIGS. 5, 6A and 6B, in order to provide more user's comfort and accurate measurements, an isolation cover 30 equipped with an inflation pouch 31 is added on the outer side of the arm rolling strip 1. The inflation pouch 31 has an internal chamber communicating with the air nozzle 111 which in turn is connected to an inflation tube 40. The isolation cover 30 further is encased by an outer cover 32, and the arm rolling strip 1 is interposed between the outer cover 32 and inflation pouch 31. The outer cover 32 has a slot 321 to allow the distal end 22 of the second rolling strip 20 to thread through. To further improve usability and product appeal, the distal end 22 may be attached to a protective cover 221. Similarly, when the relative position of the second rolling strip 20 and first rolling strip 10 is changed as shown in FIGS. 6A and 6B, the protective cover 221 can receive a user's force to extend or retract the second rolling strip 20 to pass through the slot 321 to increase or shorten the length of the arm rolling strip 1 to provide more comfort to the user and better fit.

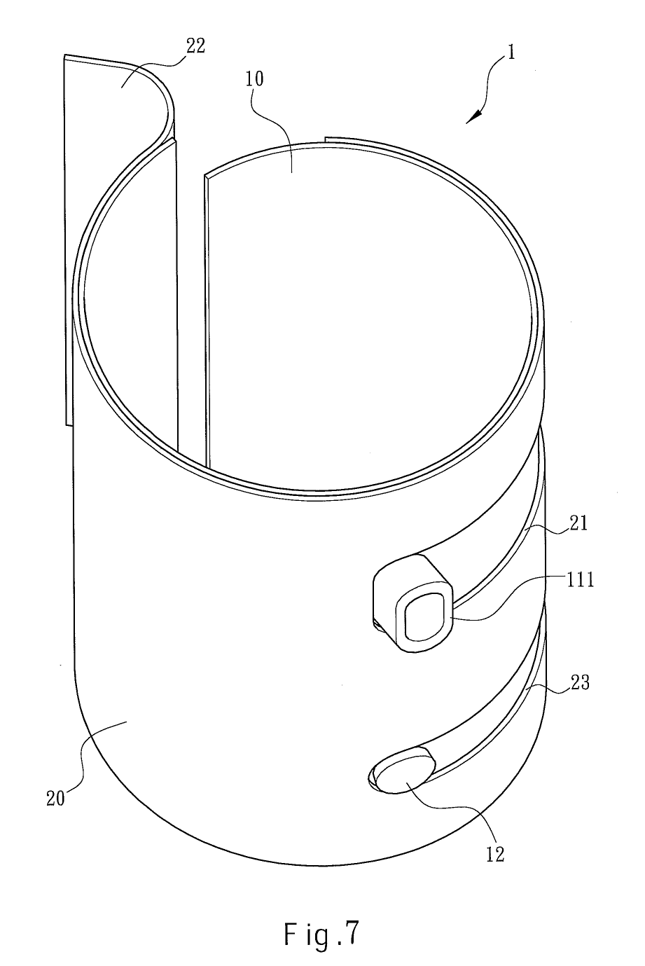

Please refer to FIG. 7 for a second embodiment in which an auxiliary track 23 is added on the arched surface of the second rolling strip 20 parallel with the guide track 21 and formed at least the same length. The first rolling strip 10 has a jutting retaining member 12 relative to the auxiliary track 23, preferably located at a position close to the distal end 22 against the auxiliary track 23 in the extended direction on the arched surface of the first rolling strip 10.

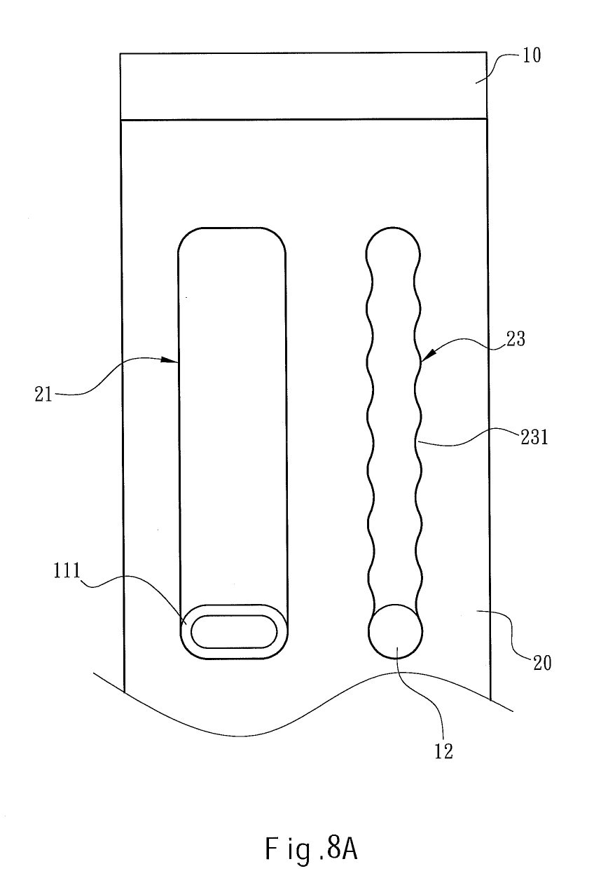

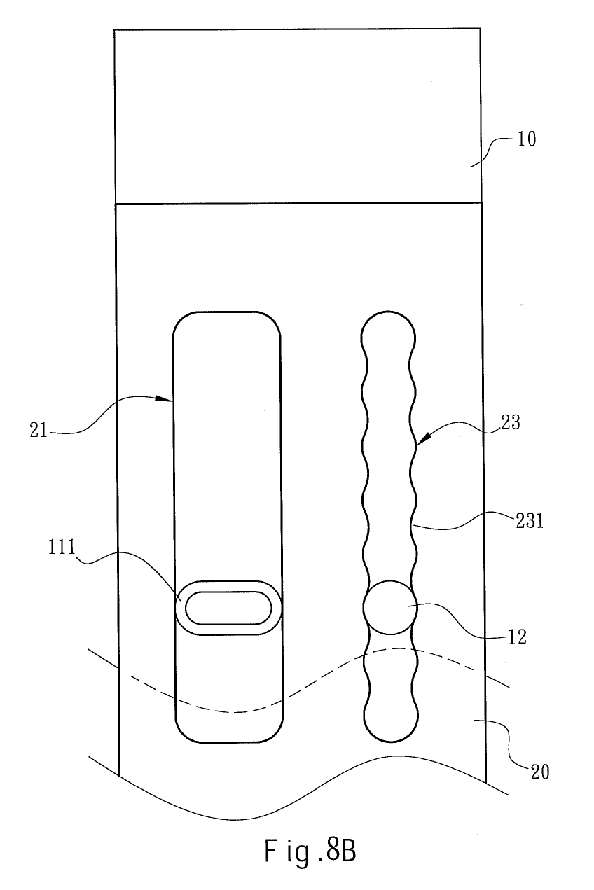

Refer to FIGS. 8A and 8B for another embodiment of the auxiliary track 23. It may have a plurality of positioning teeth 231 on at least one longer side of the auxiliary track 23 to form staged positioning when the retaining member 12 is moved on the auxiliary track 23.

What is claimed is:

1. An arm rolling strip for sphygmomanometers, comprising:
an elastic first rolling strip including an aperture to hold an air nozzle communicating therewith; and
an elastic second rolling strip which wraps partially on the first rolling strip and includes a guide track on the arched surface thereof relative to the air nozzle to restrict sliding of the second rolling strip on the arched surface of the first rolling strip.

2. The arm rolling strip of claim 1, wherein the guide track includes a plurality of positioning teeth on at least one longer side thereof.

3. The arm rolling strip of claim 1, wherein the second rolling strip includes a distal end extended in a direction opposite to the rolling direction thereof or in the tangent direction of the rolling direction thereof or any direction between the tangent direction of the rolling direction and the direction opposite to the rolling direction of the second rolling strip.

4. The arm rolling strip of claim 2, wherein the second rolling strip includes a distal end extended in a direction opposite to the rolling direction thereof or in the tangent direction of the rolling direction thereof or any direction between the tangent direction of the rolling direction and the direction opposite to the rolling direction of the second rolling strip.

5. The arm rolling strip of claim 1, wherein the second rolling strip includes an auxiliary track on the arched surface thereof parallel with the guide track and formed at a length at least equal to that of the guide track, the first rolling strip including a jutting retaining member relative to the auxiliary track to aid retaining.

6. The arm rolling strip of claim 2, wherein the second rolling strip includes an auxiliary track on the arched surface thereof parallel with the guide track and formed at a length at least equal to that of the guide track, the first rolling strip including a jutting retaining member relative to the auxiliary track to aid retaining.

7. The arm rolling strip of claim 3, wherein the second rolling strip includes an auxiliary track on the arched surface thereof parallel with the guide track and formed at a length at least equal to that of the guide track, the first rolling strip including a jutting retaining member relative to the auxiliary track to aid retaining.

8. The arm rolling strip of claim 4, wherein the second rolling strip includes an auxiliary track on the arched surface thereof parallel with the guide track and formed at a length at least equal to that of the guide track, the first rolling strip including a jutting retaining member relative to the auxiliary track to aid retaining.

9. The arm rolling strip of claim 5, wherein the auxiliary track includes a plurality of positioning teeth on at least one longer side thereof.

10. The arm rolling strip of claim 6, wherein the auxiliary track includes a plurality of positioning teeth on at least one longer side thereof.

11. The arm rolling strip of claim 7, wherein the auxiliary track includes a plurality of positioning teeth on at least one longer side thereof.

12. The arm rolling strip of claim 8, wherein the auxiliary track includes a plurality of positioning teeth on at least one longer side thereof.

13. The arm rolling strip of claim 1, wherein the first rolling strip is rolled at a gradually increasing curvature radius.

14. The arm rolling strip of claim 2, wherein the first rolling strip is rolled at a gradually increasing curvature radius.

15. The arm rolling strip of claim 5, wherein the first rolling strip is rolled at a gradually increasing curvature radius.

16. The arm rolling strip of claim 6, wherein the first rolling strip is rolled at a gradually increasing curvature radius.

17. The arm rolling strip of claim 7, wherein the first rolling strip is rolled at a gradually increasing curvature radius.

18. The arm rolling strip of claim 8, wherein the first rolling strip is rolled at a gradually increasing curvature radius.
